(19) United States
(12) Patent Application Publication
(10) Pub. No.: US 2009/0011059 A1
(43) Pub. Date: Jan. 8, 2009

(54) Ginger Extract For Inhibiting Human Drug Transporters

(76) Inventors: Naoki Ishiguro, Osaka (JP); Wataru Kishimoto, Hyogo (JP); Thomas Ebner, Ummendorf (DE); Willy Roth, Biberach (DE)

Correspondence Address: MICHAEL P. MORRIS;BOEHRINGER INGELHEIM USA CORPORATION, 900 RIDGEBURY RD, P. O. BOX 3686, RIDGEFIELD, CT, 06877-0368, US

(21) Appl. No.: 12/158,423

(22) PCT Filed: Dec. 20, 2006
(86) PCT No.: PCT/EP2006/069998

§ 371 Date: Sep. 15, 2008

(30) Foreign Application Priority Data
Dec. 22, 2005 (DE) 102005062145.7

(51) Int. Cl. A61K 36/906; A61K 31/121; A61K 31/122; A61K 31/216; A61K 31/225; A61K 31/075; A61K 31/341

(52) U.S. Cl. 424/756; 514/678; 514/543; 514/718; 514/533; 514/461; 514/690

(57) ABSTRACT

The present invention relates to the use of a ginger extract on its own or in combination with pharmaceutical compositions for inhibiting human drug transporters for positively influencing the oral bioavailability and pharmacokinetics of active substances.

The present invention relates to the use of a ginger extract on its own or in combination with pharmaceutical compositions for inhibiting human drug transporters for positively influencing the oral bioavailability and pharmacokinetics of active substances.

### BACKGROUND TO THE INVENTION

Drug transporters play an important role in the resorption, distribution and disposal in individual organs as well as the elimination of drugs. They accept a wide range of structurally different drugs. In addition to drugs, a number of endogenous substances are also substrates for these membrane-located transporters, such as e.g. bile acids, bilirubin glucuronides or phosphatidylcholine. Transporters are expressed by various cells within the body, including inter alia hepatocytes, enterocytes and kidney epithelials. They are therefore found predominantly in the liver, the intestinal wall, the kidneys and also in the capillary vessels of the blood-brain barrier. Whereas many transporters mediate the absorption of drugs into the interior of the cell, others bring about the active outward transportation (efflux) of drugs. By these functions, drug transporters influence central pharmacokinetic processes. An important role in successful pharmacotherapy is played by the function of drug transporters in the resorption of orally administered drugs from the gastrointestinal tract. Orally administered active substances may exhibit reduced resorption and hence poor bioavailability as the result of the action of an intestinal efflux transporter. When intestinal efflux processes mediated by transporters are inhibited, there is improved resorption and higher bioavailability of the drug in question. The administration of vitamin E increases e.g. the plasma exposure of cyclosporin A roughly two-fold (Yu, Dale K. The contribution of p-glycoprotein to pharmacokinetic drug-drug interactions. J. Clin. Pharmacol. 1999; 39:1203-1211). Similarly, the clinically relevant interaction between quinidine and digoxin is based on the inhibitory effects of quinidine on a drug transporter which is involved in the intestinal or renal elimination of digoxin.

Out of the large number of drug transporters which have been discovered and characterised in recent years, some have been identified as being particularly important for the transporting of drugs (cf. Table 1).

TABLE 1
Human drug transporters - expression in various organs and
examples of substrates

| | | Expression | |
|---|---|---|---|
| Name | Synonyms | Tissue/Organs | Substrate |
| MDR1 | ABCB1, P-gp | brain, intestine, liver, | paclitaxel, topotecan, diltiazem, |
| | (P-glycoprotein) | placenta | digoxin, verapamil, |
| | | | fexofenadine, loperamide, |
| | | | ritonavir |
| BCRP | ABCG2, MXR | intestine, liver, brain | daunorubicin, methotrexate |
| MRP2 | ABCC2, cMOAT, | liver, kidney, brain, | etoposide, doxorubicin, |
| | cMRP | intestine, placenta | ritonavir |
| OAT1 | SLC22A6, PAHT | kidney | zidovudine, methotrexate |
| OAT3 | SLC22A8 | kidney | ibuprofen, prostaglandin e₂ |
| OCT1 | SLC22A1 | liver, kidney, heart, | acyclovir |
| | | intestine, | |
| OCT2 | SLC22A2 | liver, kidney | amantadine, serotonin |
| OCTN1 | SLC22A4 | liver, kidney, trachea | quinidine, verapamil |
| OCTN2 | SLC22A5, SCD | kidney, heart, placenta | l-carnitine, acetylcarnitine |
| OATP-A | SLCO1A2, | liver, brain, lung, kidney | ouabain, fexofenadin |
| | SLC21A3, OATP-1 | | |
| OATP-B | OATP2B1, SLC21A9 | widely distributed | benzylpenicillin |
| OATP-C | SLCO1B1, | liver | benzylpenicillin, pravastatin, |
| | SLC21A6, LST-1, | | methotrexate, bilirubin, |
| | OATP2 | | rifampicin |
| OATP-8 | OATP1B3, SLC21A8 | liver | digoxin, ouabain, rifampicin |

It would be particularly useful, for improving pharmacokinetics after the oral administration of drugs, to be able to inhibit intestinal efflux transporters, such as e.g. P-glycoprotein, MRP-2, MRP-3 and BCRP. By improving the resorption of active substances by inhibiting the intestinal efflux transport it is possible not only to increase the bioavailability of an active substance but also to reduce the variability of the bioavailability. The latter has been found to increase as the absolute bioavailability falls. By reducing the variability of the bioavailability the therapeutic success of an oral drug therapy is decisively improved, as it is rarer for excessively high drug exposure (risk of unwanted side effects) or excessively low drug exposure (risk of failure of the treatment) to occur.

Such effects may have considerable advantages in active substance therapy. Therefore additions to pharmaceutical compositions which can increase the bioavailability of orally administered pharmaceuticals by inhibiting the intestinal efflux transport of these medicaments would be beneficial. Alternatively, influencing intestinal (uptake) transporters, such as e.g. OCTN1, OATP3, OAT3 or OCT1, could have a favourable effect on the variability of the oral pharmacokinetics. In addition to a combination of drugs with chemically defined substances for inhibiting drug transporters, ingredients present in foods or extracts of foodstuffs could also be used. These would have the advantage of being substances which could be pronounced to be safe for use in humans.

### PRIOR ART

Ginger (Zingiber officinalis) is a traditional food ingredient in many parts of the world and is also used as a phytopharmaceutical for various applications. For example, powdered ginger root is available as a preparation for preventing seasickness.

It contains about 5 to 8% of a viscous liquid balsam (oleoresin), which contains a non-steam-volatile peppery or hot fraction as well as a volatile ethereal oil fraction. The pale yellow ethereal oil makes up about 20 to 25% of the oleoresin. The composition of the ethereal oil is subject to considerable fluctuations depending on its origin. It contains as its main ingredient sesquiterpene hydrocarbons of the bisabolone type, particularly (−)-α-zingiberene and also (−)-β-sesquiphellandrene, (−)-β-bisabolene, (+)-ar-curcumene and acyclic α-farnesene (Deutsche Apothekerzeitung 1997, 137(47), 40-46).

The main component of the hot fraction, making up about 25% of the oleoresin, constitutes the homologous series of the gingerols (HagerROM 2002: Zingiberis rhizoma, Springer Verlag, Heidelberg).

### DETAILED DESCRIPTION OF THE INVENTION

Surprisingly, in vitro tests have shown that potent inhibition of various human drug transporters may be achieved by means of a ginger extract obtained by an extraction process according to the invention.

The extract obtained here is poorly soluble in hexane and differs in this characteristic from the fraction of the ethereal oil.

The process according to the invention starts from a commercially obtainable oleoresin and comprises the following steps:
(a) extracting an oleoresin with an organic solvent;
(b) extracting the combined residues from step (a) with warm water and discarding the supernatant.

In a preferred embodiment the residue thus obtained is further purified by a process comprising the steps of
(c) extracting the combined residues from step (b) with warm alcohol and
(d) concentrating the combined supernatants from step (c).

The fraction obtained in step (d) may be dissolved in an alcohol, preferably methanol or ethanol, and optionally further fractionated, for example by solid phase extraction and stepwise elution.

Non-polar organic solvents which may be used in step (a) include according to the invention low-boiling alkane solvents such as, for example, hexane, heptane, octane, pentane or cyclohexane, petrochemical distillates, propellants and solvents such as for example petrol, kerosene, petroleum ether, petroleum and other low-boiling, volatile and non-polar solvents such as for example diethyl ether, tert.-butyl-methylether, tetrahydrofuran, benzene, toluene and xylenes, while hexane is preferably used.

The alcohol used in steps (c) and (e) may be selected from among methanol, ethanol, isopropanol, n-propanol, n-butanol and other positionally isomeric butanols, n-pentanol and other positionally isomeric pentanols and may be identical or different. Preferably, methanol is used. The extraction agent in each case is used in amounts of from 4 to 10 mL/g, preferably 4 to 7 mL/g, of the oleoresin used.

The aqueous extractions are preferably carried out at a temperature of from 50 to 80° C., particularly preferably 65 to 75° C.

As an alternative to this method extractions may also be carried out with suitable aqueous organic acids or, instead of liquid-liquid extraction with organic solvents, solid phase extractions with suitable non-polar absorbents may also be carried out.

The extractions carried out in steps (a), (b) and (c) may be carried out once or several times, and the phases containing the desired product from the various extractions of one step may be combined. Preferably the extraction is carried out three times in each step and the phases containing the product are combined. The combined phases are then further processed.

A ginger extract which contains at least one compound of general formulae

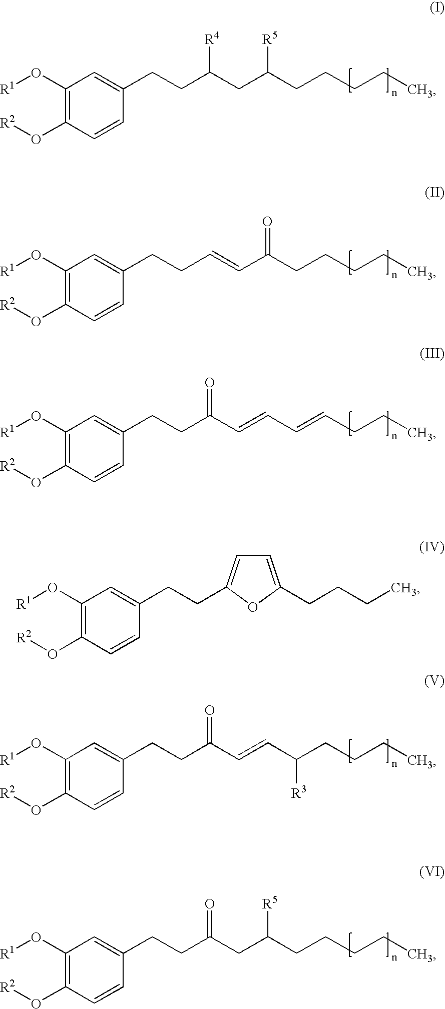

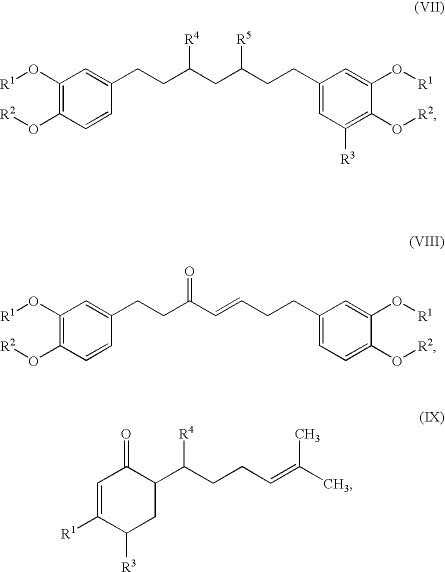

wherein
n denotes the number 1, 2 or 3,
R¹ denotes H, CH₃,
R² denotes H, CH₃,
R³ denotes H, OH, OCH₃,
R⁴ denotes H, O, OH, OCH₃, OC(O)CH₃ and
R⁵ denotes H, O, OH, OCH₃, OC(O)CH₃,
   or one of the enantiomers or diastereomers thereof, is preferred.

The following are mentioned as particularly preferred compounds of general formulae I to VI mentioned hereinbefore:

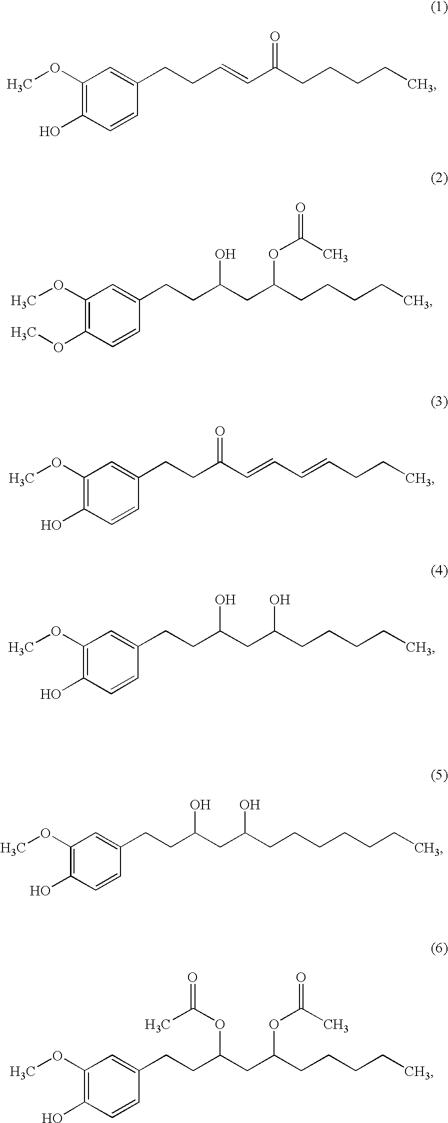

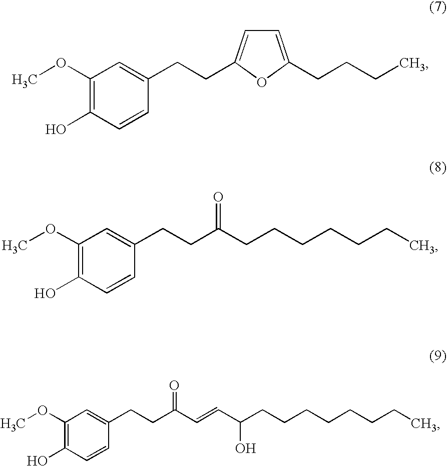

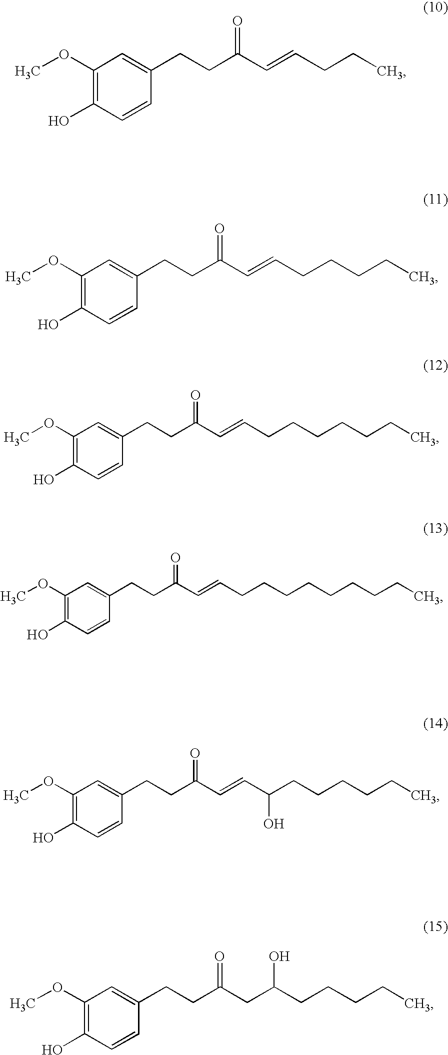

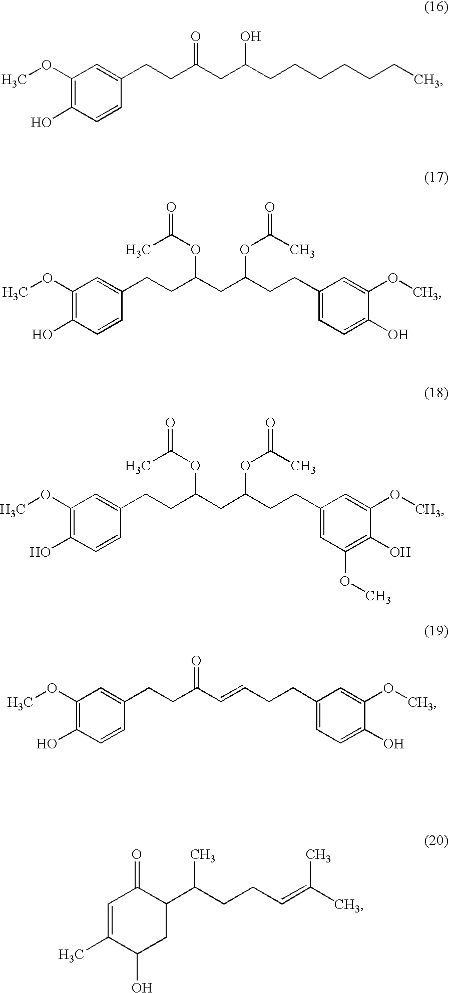

the enantiomers and the diastereomers thereof.

The compounds of general formulae I to IX were identified from the ginger extract obtained according to the invention. In order to characterise this ginger extract more precisely and establish its contents, it was suitably further purified with the aim of isolating purified fractions of individual ingredients.

In order to do this, the ginger extract obtained according to the invention was further purified by solid phase extraction on a C18 phase. The eluant of the solid phase extraction was dried out and investigated further by semipreparative high pressure liquid chromatography (HPLC). This was done by injecting fairly small aliquots of 5 to 10 mg into the semipreparative HPLC system. The eluant of the HPLC column was then collected in 60 to 65 individual fractions and each of the fractions thus obtained was investigated for its inhibitory effect on various P450 test reactions. The results of these investigations showed clearly defined zones (peaks) of higher inhibitory potency.

To clarify the chemical structure of the constituents of the individual fractions, selected samples were further purified and concentrated by repeated HPLC and then investigated by mass spectrometry and NMR spectroscopy.

Compounds (1) to (20) identified according to the invention are the typical ingredients of the non-volatile hot fraction of ginger which have already been sufficiently described in the literature. In addition to various modification products of gingerol and the various homologues thereof, a known main ingredient of ginger, [8]-gingerol (16), as well as [6]-shogaol (11), the decomposition product of the main ingredient [6]-gingerol (15) present in the largest amount, were also found.

This confirmed that the ginger extract prepared by the process described is derived from the non-volatile hot fraction and the inhibition of the drug transporters is brought about by ingredients of the gingerol type and the structural modifications and breakdown products thereof.

Many active substances have low oral bioavailability, caused by the so-called first-pass metabolism. This is the metabolic breakdown of orally administered active substances in the small intestine or in the liver, even before they are able to travel through the bloodstream to their target organ. The active substances mentioned previously, i.e. the pharmacologically active constituents of drugs, may be selected from among the drugs for acting upon the cardiovascular system in its widest sense, including those substances which serve to influence the composition of the blood (e.g. blood lipids); drugs acting on the central nervous system; drugs for treating metabolic disorders (e.g. diabetes mellitus); drugs for treating inflammatory processes in the widest sense; drugs for influencing the immune system; drugs for treating infections by bacteria, protozoa, multi-cellular parasites, viruses, fungi or prions; drugs for stopping or alleviating degenerative processes in various organs, particularly the brain, and drugs for treating cancer.

By the term “drugs” are meant substances and preparations of substances which are intended, by administration to or in the human or animal body,
1. to cure, alleviate, prevent or detect diseases, ailments, physical injury or pathological disorders;
2. to show up the nature, state or functions of the body or mental states;
3. to replace active substances or bodily fluids produced by the human or animal body;
4. to ward off, eliminate or render harmless pathogens, parasites or substances alien to the body or
5. to influence the nature, state or functions of the body or mental states.

A first object of the present invention thus relates to the use of a ginger extract for preparing a pharmaceutical composition for inhibiting drug transporters.

In a preferred first embodiment the use is characterised in that the ginger extract is prepared by a process comprising the steps of
(a) extracting an oleoresin with an organic solvent;
(b) extracting the combined residues from step (a) with warm water and discarding the supernatant.

In another preferred first embodiment the residue thus obtained is further purified by a process comprising the steps of
(c) extracting the combined residues from step (b) with warm alcohol and
(d) concentrating the combined supernatants from step (c).

In a more preferred first embodiment the ginger extract according to the invention contains at least one compound of general formulae I to IX described above, the enantiomers or diastereomers thereof.

In an even more preferred first embodiment the compounds of general formulae I to IX are selected from compounds (1) to (20) described hereinbefore, the enantiomers and the diastereomers thereof.

The drug transporters are selected from among those which are preferably found in the gastro-intestinal tract, the drug transporters OATP-8, OATP-B, OCT2, MDR-1 and BCRP having a special significance.

A second object of the present invention relates to the use of the ginger extract according to the invention in combination with a medicament for preparing a pharmaceutical composition for inhibiting drug transporters.

The drug transporters are selected from among those which are preferably found in the gastrointestinal tract, the drug transporters OATP-8, OATP-B, OCT2, MDR-1 and BCRP being particularly important.

A third object of the present invention relates to a process for preparing a pharmaceutical composition for increasing the bioavailability of an orally administered pharmaceutical compound, comprising oral administration of the pharmaceutical compound together with the ginger extract which may be obtained according to the process of the invention, to a person requiring such treatment, the ginger extract being administered in an amount which is necessary in order to increase the bioavailability of the pharmaceutical compound as compared with giving the pharmaceutical compound on its own.

The process is characterised in that the pharmaceutical compound is metabolised by a drug transporter which is preferably to be found in the gastrointestinal tract. The drug transporters selected from among OATP-8, OATP-B, OCT2, MDR-1 and BCRP are particularly preferred.

A fourth object of the present invention relates to a pharmaceutical formulation containing at least one compound of general formulae I to IX, the enantiomers or the diastereomers thereof, optionally together with one or more inert carriers and/or diluents.

In a preferred embodiment the pharmaceutical formulation contains at least one compound of the previously mentioned formulae (1) to (20), the enantiomers or the diastereomers thereof.

A fifth object of the present invention relates to a pharmaceutical composition consisting of two or more components which are optionally physically separate from one another, comprising:
(a) a first component consisting of the ginger extract which may be obtained according to one of claims 2 to 8, and one or more pharmaceutically acceptable diluents and/or carriers; and
(b) a second component containing a pharmaceutical composition, comprising a pharmaceutical compound which is metabolised by drug transporters, and one or more pharmaceutically acceptable diluents and/or carriers.

A preferred fifth embodiment is characterised in that the first component contains at least one compound of the previously mentioned formulae I to IX, the enantiomers or diastereomers thereof.

The compounds of the previously mentioned formulae (1) to (20) are of exceptional importance.

A more preferred fifth embodiment is characterised in that the pharmaceutical compound of the second component is metabolised by the drug transporters which are selected from among OATP-8, OATP-B, OCT2, MDR-1 and BCRP.

### EXPERIMENTAL SECTION

10 g of an oleoresin (Eramex Aromatics GmbH) are extracted three times with 50 mL hexane and the supernatant (organic phase) is discarded. The residues are combined and extracted three times with 40 mL of water heated to 70° C. The supernatant is discarded again and the combined residues are extracted three times with 40 mL of methanol heated to 70° C. The residue is discarded. The supernatant obtained is concentrated by rotary evaporation and dissolved in methanol again.

Diagram 1 shows an overview of the extraction process of the ginger extract according to the invention of the oleoresin with separation of the ethereal oils.

The ginger extract thus obtained was then purified further by solid phase extraction on a C18 phase. The eluant of the solid phase extraction was dried and investigated further by semipreparative high pressure liquid chromatography (HPLC). For this, small aliquots of 5 to 10 mg were injected into the semipreparative HPLC system. The eluant of the HPLC column was then collected in 60 to 65 individual fractions and each of the fractions thus obtained was investigated for its inhibitory effect on various drug transporters.

### Formulations

Concentrations <100 μg/ml of the ginger extract are sufficient for exerting the desired effects on intestinal drug transporters (cf. FIGS. 1 to 6). With an intestinal liquid volume (fasting) of 250 ml-500 ml, 25-50 mg of the ginger extract would be needed for each individual dose of oral preparation. As the intestinal liquid volume is increased by food intake, a larger amount of about 100 mg-125 mg of the ginger extract would be needed if the drug was taken after or during a meal.

The prerequisite for the inhibition of drug transporters is that the ginger extract is largely dissolved in the intestinal tract. As the ginger extract is very poorly soluble in water, it was necessary to develop formulations that dissolved well in aqueous media. For this reason, formulation screening was carried out with a variety of pharmaceutically conventional excipients. More extensive tests were then carried out with particularly suitable excipients to obtain formulations which were optimum in terms of both quality and quantity.

1. Selection of Functional Excipients with a High Supersaturation of Ginger Extracts in Aqueous Media
1.1 Preparation of Formulations with Meltable Excipients

30 mg ginger extract were heated, with stirring, with 60 mg of the excipient in an aluminium plate with round depressions to a temperature which was about 5° C. above the melting temperature of the excipient, and mixed with thorough stirring. Then the mixture was cooled with stirring until a solid preparation was obtained. This was then used directly for in vitro testing.

1.2 Preparation of Formulations with Non-Meltable Excipients

200 mg ginger extract were dissolved in 2 ml alcohol, preferably ethanol. Then 300 μl in each case were intensively mixed with 60 mg of the excipient, and then the alcohol was evaporated off. The solid form obtained was then used directly for in vitro testing.

### 1.3 Results of the In Vitro Releases

### Method:

30 mg formulation, which contained 10 mg ginger extract in each case, were stirred into 20 ml of water at a temperature of 37° C. and the release was determined after 2, 5, 10, 15, 20, 25 and 30 minutes by UV measurement at 280 and 358 nm. This active substance charge corresponds to a human dose of 100 mg, which is taken together with 200 ml of water. Unformulated ginger extract was used as a reference substance. The releases were calculated from the quotient of the extinctions of the respective formulations and the extinctions of the reference form.

Table 2 provides an overview of the most important results.

TABLE 2
Overview of maximum releases of various formulations in water at
37° C.

| | release | supersaturation | |
|---|---|---|---|
| excipient | [%] | factor | suitability |
| without | 4.5 | 1.0 | |
| tartaric acid | 2.4 | 0.5 | no |
| Cutina HR | 3.0 | 0.7 | no |
| Gelucire 50/13 | 6.0 | 1.3 | no |
| Stearylalcohol | 6.0 | 1.3 | no |
| Precirol ATO5 | 7.0 | 1.6 | no |
| Suppocire AM | 13.0 | 2.9 | conditional |
| Gelucire 39/01 | 13.0 | 2.9 | conditional |
| Gelucire 43/01 | 13.0 | 2.9 | conditional |
| Labrafil M1944 | 15.0 | 3.3 | conditional |
| Meglumin | 25.3 | 5.6 | good |
| PEG 6000 | 54.0 | 12.0 | good |
| Cremophor EL | 83.0 | 18.4 | good |
| Poloxamer 188 | 98.0 | 21.8 | good |
| Gelucire 44/14 | 100.0 | 22.2 | good |

It is apparent that even at the selected high active substance load of 33% with the most suitable formulations the ginger extract was released completely, corresponding to a more than 20-fold supersaturation. Good resorption of the ginger extract is thus ensured

2. Formulation Examples for Combinations of Ginger Extract and Active Substances the Bioavailability of which can be Increased Using Ginger Extract

In each case combinations of the ginger extract according to the invention and a pharmaceutical substance whose bioavailability is to be increased are used. This may be done using various formulation approaches:
(i) Ginger extract and active substance are formulated separately and then administered simultaneously or with a short interval between taking the ginger formulation and the formulation of the pharmaceutical composition (in order to achieve the saturation of drug transporters beforehand).
(ii) Semi-finished products of the ginger extract and active substance are prepared, which are then further processed to make a final fixed combination, the release of the ginger extract and medicament being matched to one another. This can be done, for example, by compressing a granulated ginger preparation and granulated pharmaceutical composition with tabletting excipients to produce a tablet, or multiparticulate formulations of ginger extract and medicament are together packed into a capsule.
(iii) Ginger extract and medicament are formulated together as a fixed medicament combination.

Some examples of the various types of formulation will now be described, which provide an illustration of the scope of the invention without limiting the overall scope.

### Example 1

### Preparation of a Ginger Formulation by Embedding the Ginger Extract in a Melt of Gelucire 44/14 and Packing into Hard Capsules

20 g ginger extract and 40 g Gelucire 44/14 are stirred at 50° C. until a homogeneous melt is formed. Using a liquid fill apparatus 75 mg of the melt obtained is transferred with stirring into a hard #5 capsule (gelatine or HPLMC). This corresponds to a dosage of 25 mg ginger extract. Analogously, 150 mg of melt (corresponding to a dosage of 50 mg ginger extract) may be packed into a #4 capsule, 225 mg of melt (corresponding to a dosage of 75 mg ginger extract) into a #2 capsule, 300 mg of melt (corresponding to a dosage of 100 mg ginger extract) into a #2el capsule or 450 mg of melt (corresponding to a dosage of 150 mg ginger extract) into a #0 capsule.

### Example 2

### Preparation of a Ginger Formulation by Embedding the Ginger Extract in a Melt of Poloxamer 188 and Transferring into Hard Capsules

20 g ginger extract and 40 g Poloxamer 188 are stirred at 64° C. until a homogeneous melt is formed. Using a liquid fill apparatus 75 mg of the melt obtained are transferred into a hard capsule #5 (gelatine or HPLMC) with stirring. This corresponds to a dosage of 25 mg ginger extract. Analogously 150 mg of melt (corresponding to a dosage of 50 mg ginger extract) may be transferred into a #4 capsule, 225 mg of melt (corresponding to a dosage of 75 mg ginger extract) into a #2 capsule, 300 mg of melt (corresponding to a dosage of 100 mg ginger extract) into a #2el capsule or 450 mg of melt (corresponding to a dosage of 150 mg ginger extract) into a #0 capsule.

### Example 3

### Preparation of a Ginger Formulation by Embedding the Ginger Extract in a Melt in Cremophor EL and Transferring into Hard Capsules

20 g ginger extract, 40 g Cremophor EL and 15 g microcrystalline cellulose are stirred at 48° C. until a homogeneous melt is formed in which the microcrystalline cellulose is uniformly suspended. Using a liquid fill apparatus 93.8 mg of the melt obtained are transferred with stirring into a #5 hard capsule (gelatine or HPLMC). This corresponds to a dosage of 25 mg ginger extract. Analogously 187.5 mg of melt (corresponding to a dosage of 50 mg ginger extract) may be transferred into a #4 capsule, 281.3 mg of melt (corresponding to a dosage of 75 mg ginger extract) into a #1 capsule, 375 mg of melt (corresponding to a dosage of 100 mg ginger extract) into a #1 capsule or 562.5 mg of melt (corresponding to a dosage of 150 mg ginger extract) into a #0 capsule.

### Example 4

### Preparation of a Ginger Formulation by Embedding the Ginger Extract in a Melt in PEG 6000 and Transferring into Hard Capsules

20 g ginger extract and 40 g PEG 6000 are stirred at 67° C. until a homogeneous melt is formed. Using a liquid fill apparatus 75 mg of the melt obtained is transferred with stirring into a hard capsule #5 (gelatine or HPLMC). This corresponds to a dosage of 25 mg ginger extract. Analogously 150 mg of melt (corresponding to a dosage of 50 mg ginger extract) may be packed into a #4 capsule, 225 mg of melt (corresponding to a dosage of 75 mg ginger extract) into a #2 capsule, 300 mg of melt (corresponding to a dosage of 100 mg ginger extract) into a #2el capsule or 450 mg of melt (corresponding to a dosage of 150 mg ginger extract) into a #0 capsule.

### Example 5

### Preparation of a Ginger Formulation by Embedding the Ginger Extract in a Melt in PEG 6000/Poloxamer 188 and Transferring into Hard Capsules

20 g ginger extract, 20 g Poloxamer 188 and 20 g PEG 6000 are stirred at 67° C. until a homogeneous melt is formed. Using a liquid fill apparatus 75 mg of the melt obtained are packed with stirring into a hard capsule #5 (gelatine or HPLMC). This corresponds to a dosage of 25 mg ginger extract. Analogously 150 mg of melt (corresponding to a dosage of 50 mg ginger extract) may be packed into a #4 capsule, 225 mg of melt (corresponding to a dosage of 75 mg ginger extract) into a #2 capsule, 300 mg of melt (corresponding to a dosage of 100 mg ginger extract) into a #2el capsule or 450 mg of melt (corresponding to a dosage of 150 mg ginger extract) into a #0 capsule.

### Example 6

### Preparation of a Ginger Formulation by Extrusion of the Ginger Extract with Poloxamer 188 and Transferring into Hard Capsules

20 g ginger extract and 40 g Poloxamer 188 are mixed dry and at 52° C. extruded in a 16 mm twin-screw extruder with a 1 mm die plate and head removal. The roughly 1 mm long cylinders formed are rounded off in a spheronizer at about 51° C. and then using a capsule filling machine packed into hard capsules (gelatine or HPLMC). If 75 mg are packed into a #5 capsule, this corresponds to a dosage of 25 mg ginger extract. Analogously 150 mg extrudate (corresponding to a dosage of 50 mg ginger extract) may be packed into a #4 capsule, 225 mg extrudate (corresponding to a dosage of 75 mg ginger extract) into a #2 capsule, 300 mg extrudate (corresponding to a dosage of 100 mg ginger extract) into a #2el capsule or 450 mg extrudate (corresponding to a dosage of 150 mg ginger extract) into a #0 capsule.

### Example 7

### Preparation of a Ginger Formulation by Extrusion of the Ginger Extract with Peg 6000 and Transferring into Hard Capsules

20 g ginger extract and 40 g PEG 6000 are mixed dry and at 55° C. extruded in a 16 mm twin-screw extruder with a 1 mm die plate and head removal. The roughly 1 mm long cylinders formed are rounded off in a spheronizer at about 51° C. and then using a capsule filling machine packed into hard capsules (gelatine or HPLMC). If 75 mg are packed into a #5 capsule, this corresponds to a dosage of mg ginger extract. Analogously 150 mg extrudate (corresponding to a dosage of 50 mg ginger extract) may be packed into a #4 capsule, 225 mg extrudate (corresponding to a dosage of 75 mg ginger extract) into a #2 capsule, 300 mg extrudate (corresponding to a dosage of 100 mg ginger extract) into a #2el capsule or 450 mg extrudate (corresponding to a dosage of 150 mg ginger extract) into a #0 capsule.

### Example 8

### Preparation of a Ginger Formulation by Extrusion of the Ginger Extract with Poloxamer 188/PEG 6000 and Transferring into Hard Capsules

20 g ginger extract, 20 g Poloxamer 188 and 20 g PEG 6000 188 are mixed dry and at 53° C. extruded in a 16 mm twin-screw extruder with a 1 mm die plate and head removal. The roughly 1 mm long cylinders formed are rounded off in a spheronizer at about 51° C. and then using a capsule filling machine packed into hard capsules (gelatine or HPLMC). If 75 mg are packed into a #5 capsule, this corresponds to a dosage of 25 mg ginger extract. Analogously 150 mg extrudate (corresponding to a dosage of 50 mg ginger extract) may be packed into a #4 capsule, 225 mg extrudate (corresponding to a dosage of 75 mg ginger extract) into a #2 capsule, 300 mg extrudate (corresponding to a dosage of 100 mg ginger extract) into a #2el capsule or 450 mg extrudate (corresponding to a dosage of 150 mg ginger extract) into a #0 capsule.

### Example 9

### Preparation of a Ginger Formulation by Extrusion of the Ginger Extract with Poloxamer 188/PEG 6000 and Transferring into Hard Capsules

20 g ginger extract, 40 g Cremophor EL and 20 g microcrystalline cellulose are intensively mixed and at 50° C. extruded in a 16 mm twin-screw extruder with a 1 mm die plate and head removal. The roughly 1 mm long cylinders formed are rounded off in a spheronizer at about 51° C. and then using a capsule filling machine packed into hard capsules (gelatine or HPLMC). If 100 mg are packed into a #4 capsule, this corresponds to a dosage of 25 mg ginger extract. Analogously 200 mg extrudate (corresponding to a dosage of 50 mg ginger extract) may be packed into a #3 capsule, 300 mg extrudate (corresponding to a dosage of 75 mg ginger extract) into a #2el capsule, 400 mg extrudate (corresponding to a dosage of 100 mg ginger extract) into a #1el capsule or 600 mg extrudate (corresponding to a dosage of 150 mg ginger extract) into a #0el capsule.

The capsules obtained according to Examples 1 to 9 are used as free combinations with standard commercial formulations of active substances, the availability of which is reduced by drug transporters.

TABLE 3
Examples of active substances which are substrates for drug
transporters

| | oral availability |
|---|---|
| active substance | [%] |
| Raloxifene | 2 |
| Lovastatin | 4 |
| Saquinavir | 4 |
| Docetaxel | 8 |
| Sirolimus | 15 |
| Tacrolimus | 16 |
| Verapamil | 17 |
| Cyclosporin A | 27 |
| Fexofenadine | 30 |
| Loperamide | 40 |
| Topotecan | 42 |
| Venlafaxine | 43 |

The medicament forms are taken either simultaneously or with a time delay of about 15 minutes between taking the ginger formulation and the active substance of reduced availability; taking the active substance after a time delay is preferable as it ensures that the transporter systems are saturated.

### 3. Pellet Formulations for Active Substances which are Substrates for Drug Transporters

### Example 10

### Preparation of a Simvastatin Formulation by Wet Extrusion of Simvastatin with Microcrystalline Cellulose

20 g simvastatin, 2 g citric acid and 18 g microcrystalline cellulose are mixed dry and extruded at ambient temperature and with the simultaneous addition of water in a 16 mm twin-screw extruder with a 0.8 mm die plate. The extruded strips produced are broken up and rounded off in a spheronizer at ambient temperature and then dried.

### Example 11

### Preparation of a Lovastatin-Formulation by Wet Extrusion of Lovastatin with Microcrystalline Cellulose

20 g lovastatin and 30 g microcrystalline cellulose are mixed dry and at ambient temperature and extruded at ambient temperature and with the simultaneous addition of water in a 16 mm twin-screw extruder with a 0.8 mm die plate. The extruded strips produced are broken up and rounded off in a spheronizer at ambient temperature and then dried.

### Example 12

### Preparation of a Verapamil-Formulation by Wet Extrusion of Verapamil with Microcrystalline Cellulose

75 g verapamil and 25 g microcrystalline cellulose are mixed dry and extruded at ambient temperature and with the simultaneous addition of water in a 16 mm twin-screw extruder with a 0.8 mm die plate. The extruded strips produced are broken up and rounded off in a spheronizer at ambient temperature and then dried.

All the active substances of Tables 2 and 3 may be formulated analogously, the ratio of active substance: microcrystalline cellulose being selected so that the entire formulation is in the range from 100 to 300 mg, i.e. with low doses of active substances (<=40 mg active substance) 40 to 80% cellulose are used, while with higher doses of active substances only the minimum amount of >=20% cellulose needed for good workability is used. If desired other excipients may also be added to the formulations, for improving solubility and/or for stabilisation.

### Example 13

### Fixed Pharmaceutical Combinations of Non-Delayed Release Active Substance Pellets of Drug Transporters and Ginger Formulations in Capsules

By weighing out the respective pellet formulations in amounts which correspond to the desired dosages in each case, it is very easy to achieve all the desired combinations.

Some Examples are given in Table 4.

TABLE 4
Examples of different active substance combinations

| | | fill amounts | | | fill amounts of |
|---|---|---|---|---|---|
| | | of pellets of | ginger | | pellets of |
| active substance | dose of active | active | formulation | | ginger |
| formulation according | substance | substance | example; dose | | formulation |
| to Example 11 | [mg] | [mg] | of ginger [mg] | | [mg] |
| Simvastatin Example 10 | 20 | 40 | 2 | 25 | 75 |
| Simvastatin Example 10 | 80 | 160 | 2 | 100 | 300 |
| Lovastatin Example 11 | 40 | 100 | 2 | 75 | 225 |
| Verapamil Example 12 | 80 | 106.4 | 2 | 100 | 300 |

### Example 14

### Pellet Formulations with Delayed Release of Active Substances, which are Substrates for Drug Transporters

100 g pellets of Examples 10 to 12 or analogous pellets of examples of other active substances are coated with a retardant coating of the following composition in a Hüttlin-Coater Microlab or other suitable apparatus:

| | | |
|---|---|---|
| hydroxypropylmethylcellulose-phthalate | 2.25 | g |
| Eudragit S | 0.25 | g |
| talc | 0.5 | g |
| triethylcitrate | 0.5 | g |
| isopropanol | 50 | ml |

After drying, pellets are obtained from which the active substance is only released after a lag time of about 15 minutes. Combining them with non-delayed release pellets ensures that drug transporters are already inhibited when the active substance is flooding in.

### Example 15

### Fixed Pharmaceutical Combinations of Delayed Release Active Substance Pellets of Drug Transporters and Ginger Formulations in Capsules

By weighing out the respective pellet formulations in amounts which correspond to the desired dosages in each case, it is very easy to achieve all the desired combinations.

Some Examples are given in Table 5.

TABLE 5
Examples of different active substance combinations

| | | fill amount of | | | |
|---|---|---|---|---|---|
| | dose of | pellets of | ginger | | fill amount of |
| formulation of active | active | active | formulation | | pellets of |
| substance according to | substance | substance | example; dose | | ginger |
| Example 11 | [mg] | [mg] | of ginger | | formulation |
| Simvastatin Example 10 | 20 | 41.3 | 2 | 25 | 75 |
| Simvastatin Example 10 | 80 | 165 | 2 | 100 | 300 |
| Lovastatin Example 11 | 40 | 103.5 | 2 | 75 | 225 |
| Verapamil Example 12 | 80 | 109.7 | 2 | 100 | 300 |

### Example 16

### Fixed Pharmaceutical Combinations of (Non)Delayed-Release Active Substance Pellets of Substrates of Drug Transporters and Ginger Formulations in Tablets

The ginger-containing pellets in Examples 6 to 9 and the active substance-containing pellets in Examples 10 to 12 can also be compressed into tablets with excipients suitable for tablet-making:

Thus, for example, 40 g of pellets from Example 10 and 75 g pellets from Example 4 are mixed with 120 g microcrystalline cellulose, 40 g lactose and 7.5 g AcDiSol and 2.5 g magnesium stearate and compressed into tablets with a total weight of 285 mg, containing 20 mg simvastatin and 25 mg ginger extract.

Many other combinations of Examples 6 to 9 and 10 to 12 may be prepared analogously.

### Example 17

### Fixed Pharmaceutical Combinations of Non-Delayed-Release Active Substance Pellets of Substrates of Drug Transporters and Ginger Formulations in Tablets

After extrusion the ginger-containing pellets of Examples 6 to 9 are ground up using suitable mills, e.g. centrifugal mills, to form granules with particle sizes in the range from 100 to 500 μm, and similarly the active substance-containing extrudates of Examples 10 to 12, which are ground up after drying. The granules can be compressed with excipients suitable for tabletting.

Thus, for example, 40 g of ground granules from Example 10, 75 g ground granules from Example 4 are mixed with 80 g microcrystalline cellulose, 20 g Lactose and 5 g AcDiSol and 1.5 g magnesium stearate and compressed into tablets with a total weight of 219.5 mg, containing 20 mg simvastatin and 25 mg ginger extract.

Many other combinations of Examples 6 to 9 and 10 to 12 may be prepared analogously.

### BRIEF DESCRIPTION OF THE FIGURES

FIG. 1 shows an overview of the extraction process of the ginger extract according to the invention from an oleoresin with separation of the ethereal oils.

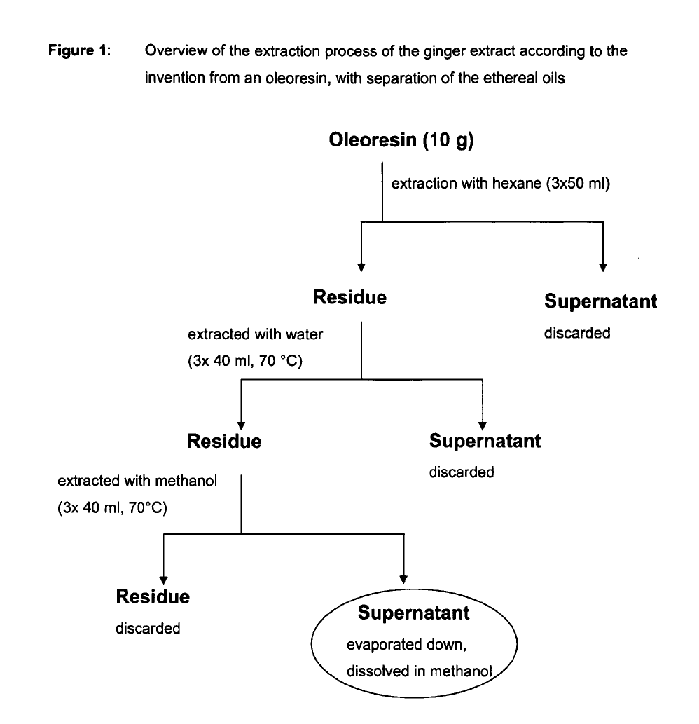

FIG. 2 shows the inhibition of the OATP-8 mediated oestradiol-17β-glucuronide uptake by the ginger extract according to the invention.

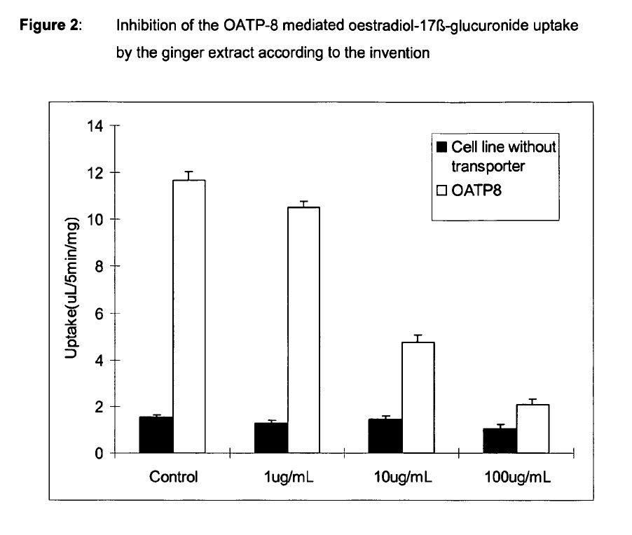

FIG. 3 shows the inhibition of the OATP-B mediated oestron sulphate uptake by the ginger extract according to the invention.

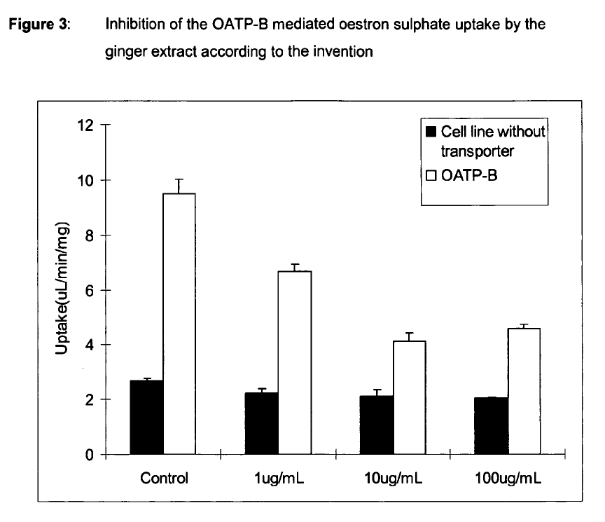

FIG. 4 shows the inhibition of the OCT2 mediated tetraethylammonium uptake by the ginger extract according to the invention.

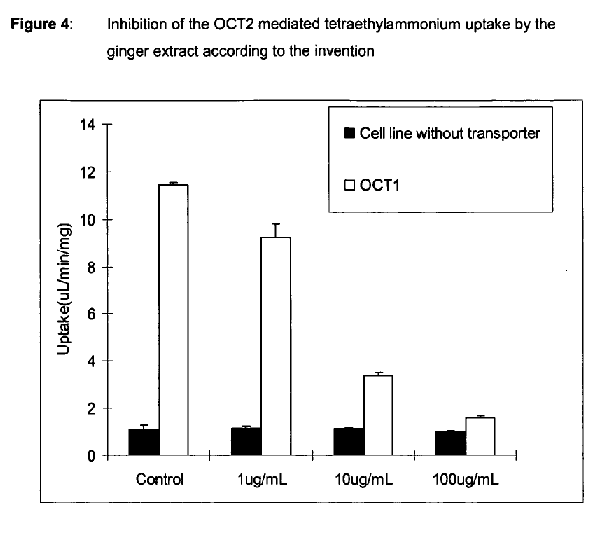

FIG. 5 shows the inhibition of the MDR-1 mediated oestradiol-17β-glucuronide uptake by the ginger extract according to the invention.

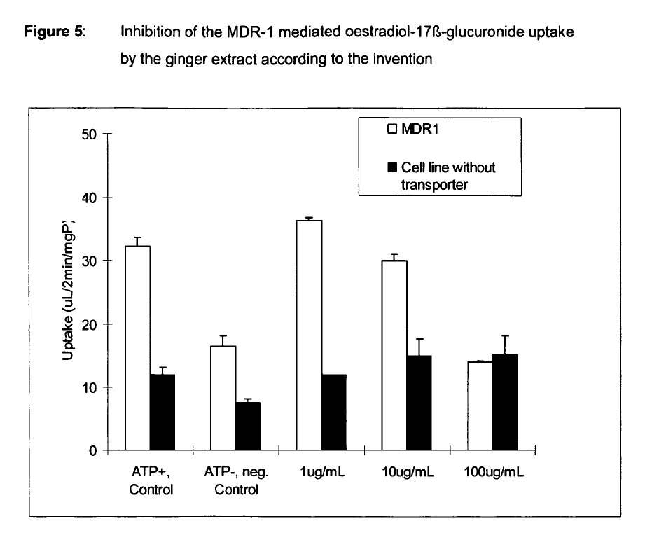

FIG. 6 shows the inhibition of the BCRP mediated oestron sulphate uptake by the ginger extract according to the invention.

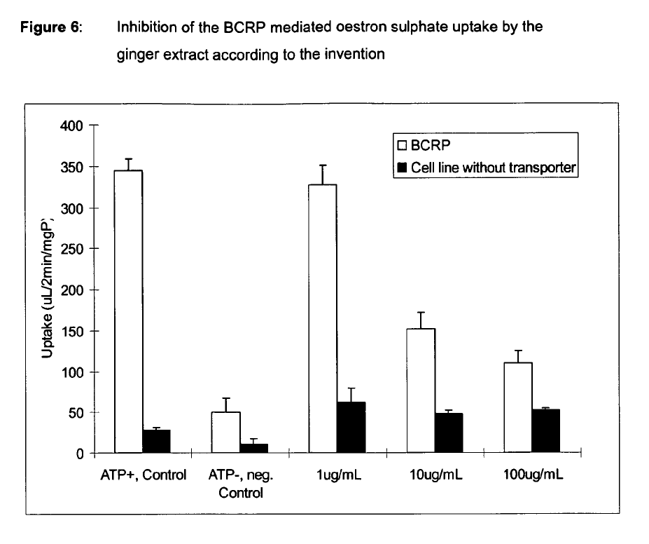

1-25. (canceled)

26. A method of inhibiting a drug transporter comprising administering to an individual a ginger extract.

27. The method according to claim 26, wherein the ginger extract is produced by a process comprising the steps of
(a) extracting an oleoresin with a non-polar organic solvent;
(b) extracting the combined residues from step (a) with warm water and discarding the supernatant.

28. The method according to claim 27, wherein the combined residues obtained in step (b) are further purified by a process comprising the steps of
(c) extracting with warm alcohol and
(d) concentrating the combined residues from step (c).

29. The method according to claim 27, wherein the in non-polar organic solvent in step (a) is selected from the groups consisting of a low-boiling alkane solvent, a petrochemical distillate, a propellant or another low-boiling, volatile and non-polar solvent.

30. The method according to claim 27, wherein the non-polar organic solvent in step (a) is hexane.

31. The method according to claim 28, wherein the alcohol in step (c) is selected from the group consisting of a methanol, ethanol, isopropanol, n-propanol, n-butanol or another positionally isomeric butanol, n-pentanol, another positionally isomeric pentanol, and mixtures thereof.

32. The method according to claim 28, wherein the alcohol in step (c) is methanol.

33. The method according to claim 27, wherein the extraction agent used in steps (a), (b) and (c) is used in each case in amounts of 4 to 10 mL/g of the oleoresin used.

34. A method of inhibiting a drug transporter comprising the administration of a ginger extract, wherein the ginger extract contains at least one compound of general formulae 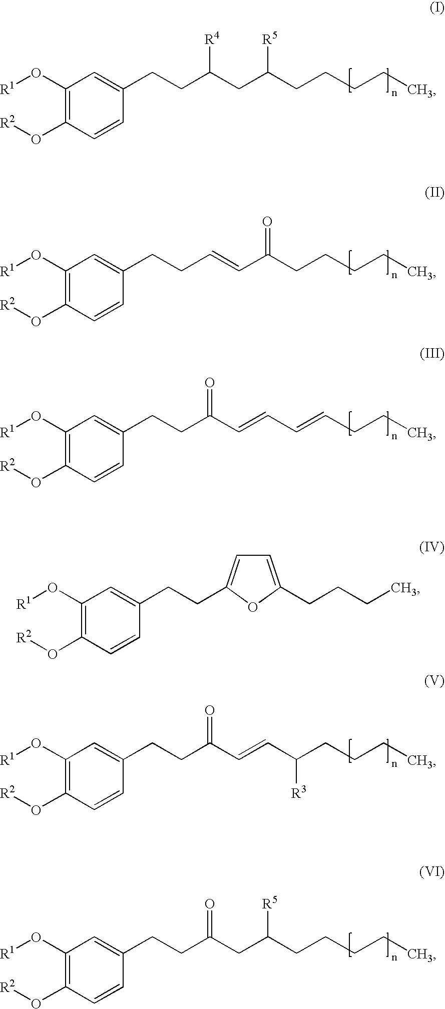 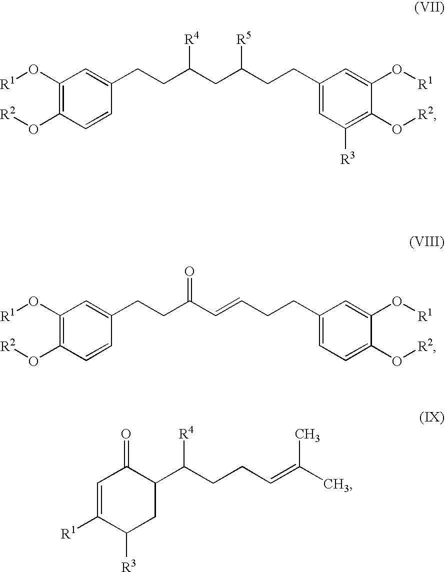
wherein
n denotes the number 1, 2 or 3,
R¹ denotes H, CH₃,
R² denotes H, CH₃,
R³ denotes H, OH, OCH₃,
R⁴ denotes H, O, OH, OCH₃, OC(O)CH₃ and
R⁵ denotes H, O, OH, OCH₃, OC(O)CH₃; and
wherein each general formula represents a compound, an enantiomer of the compound or a diastereomer of the compound.

35. The method according to claim 34, wherein the compound of general formulae I to IX is selected from the group consisting of 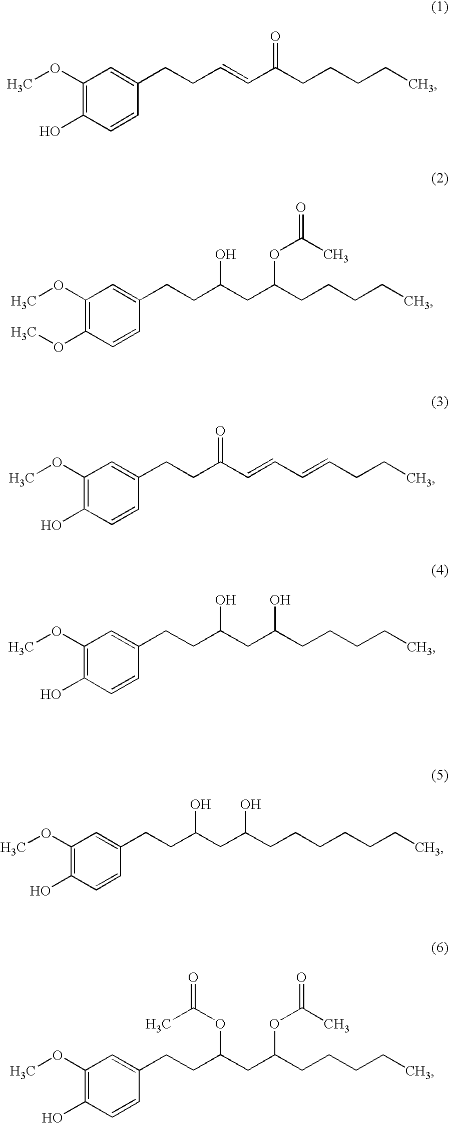 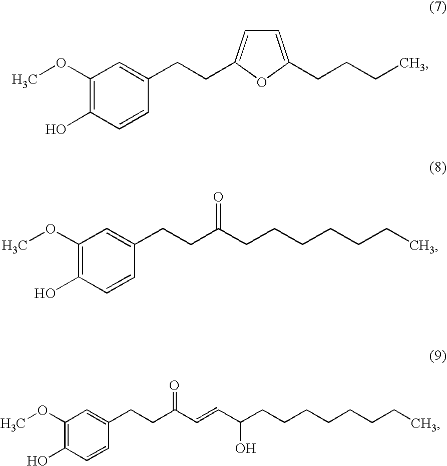 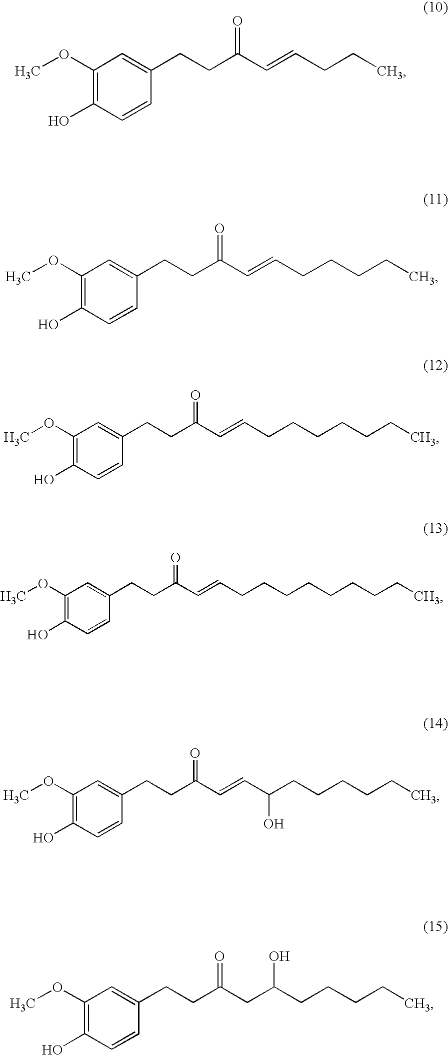 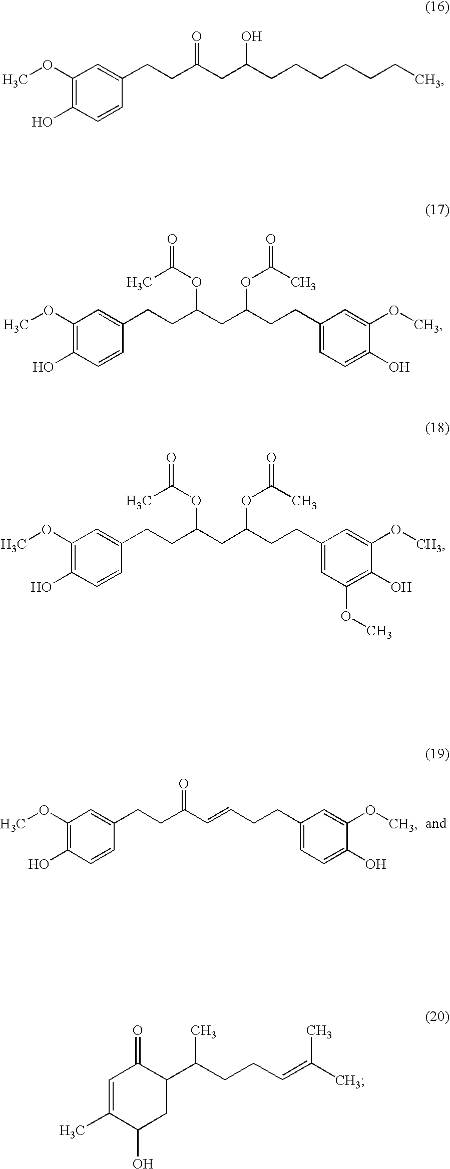
wherein each structural formula represents a compound, an enantiomer of the compound or a diastereomer of the compound.

36. The method according to claim 26, wherein the drug transporter is selected from among those which are preferably to be found in the gastro-intestinal tract.

37. The method according to claim 26, wherein the drug transporter is selected from the group consisting of OATP-8, OATP-B, OCT2, MDR-1 and BCRP.

38. The method according to claim 26, wherein the ginger extract is in combination with a medicament.

39. The method according to claim 38, wherein the drug transporter is selected from among those which are preferably to be found in the gastro-intestinal tract.

40. The method according to claim 38, wherein the drug transporter is selected from the group consisting of OATP-8, OATP-B, OCT2, MDR-1 and BCRP.

41. A process for increasing the bioavailability of an orally administered pharmaceutical compound, comprising orally administering to a person requiring such treatment a pharmaceutical compound together with the ginger extract obtained according to claim 27, the ginger extract being administered in an amount which is necessary in order to increase the bioavailability of the pharmaceutical compound as compared with administering the pharmaceutical compound on its own.

42. The process according to claim 41, wherein the pharmaceutical compound is metabolised by a drug transporter.

43. The process according to claim 41, wherein the pharmaceutical compound is metabolised by a drug transporter which is preferably to be found in the gastro-intestinal tract.

44. A process according to claim 41, wherein the pharmaceutical compound is transported by a drug transporter which is selected from the group consisting of OATP-8, OATP-B, OCT2, MDR-1 and BCRP.

45. A pharmaceutical formulation containing at least one compound of general formula I to IX, wherein each general formula represents a compound, an enantiomer of the compound or a diastereomer of the compound, optionally together with one or more inert carriers and/or diluents.

46. A pharmaceutical formulation containing at least one compound of structural formulae (1) to (20), wherein each structural formula represents a compound, an enantiomer of the compound or a diastereomer of the compound, optionally together with one or more inert carriers and/or diluents.

47. A pharmaceutical composition consisting of two or more components which are optionally physically separate from one another, comprising:
(a) a first component consisting of the ginger extract which may be obtained according to claim 27 and one or more pharmaceutically acceptable diluents and/or carriers; and
(b) a second component containing a pharmaceutical composition, comprising a pharmaceutical compound which is metabolised by drug transporters, and one or more pharmaceutically acceptable diluents and/or carriers.

48. The pharmaceutical composition according to claim 47, wherein the first component contains at least one compound general formulae I to IX.

49. The pharmaceutical composition according to claim 47, wherein the first component contains at least one compound general formulae (1) to (20).

50. The pharmaceutical composition according to one of claim 47, wherein the pharmaceutical compound of the second component is metabolised by the drug transporters which are selected from the group consisting of OATP-8, OATP-B, OCT2, MDR-1 and BCRP.
